(19) United States
(12) Patent Application Publication
(10) Pub. No.: US 2024/0387015 A1
(43) Pub. Date: Nov. 21, 2024

(54) MANAGING PRESCRIPTION MONITORING PROGRAM DATA

(71) Applicant: VETSNAP CORP., Thousand Oaks, CA (US)

(72) Inventors: Steven Patrick ELLIOTT, Thousand Oaks, CA (US); Yangyang SHI, Thousand Oaks, CA (US)

(21) Appl. No.: 18/669,010

(22) Filed: May 20, 2024

Related U.S. Application Data
(60) Provisional application No. 63/467,807, filed on May 19, 2023.

(51) Int. Cl. G16H 15/00

(52) U.S. Cl. CPC G16H 15/00

(57) ABSTRACT

Technologies for managing prescription drug monitoring program (PMP) data include receiving a request to generate a report of PMP data maintained by a medical practice, the report to be submitted to a regulatory entity system of a given state. One or more data requirements for the report associated with the state are identified. One or more records including data that complies with the one or more data requirements is obtained from a digital controlled substance logbook. The data is extracted from the one or more records, and the PMP report is generated from the extracted data.

### CROSS-REFERENCE TO RELATED APPLICATIONS

This patent application claims priority to U.S. Provisional Patent Application Ser. No. 63/467,807, entitled “SYSTEMS AND TECHNIQUES FOR MANAGING PRESCRIPTION MONITORING PROGRAM DATA,” filed May 19, 2023. The disclosure set forth in the referenced application is incorporated herein by reference in its entirety.

### FIELD

Embodiments disclosed herein generally relate to practice management and inventory systems, and more specifically, to techniques for managing prescription drug program (PMP) data.

### BACKGROUND

A “prescription monitoring program” (PMP) (also referred to as a “prescription monitoring drug program” (PDMP)) is a database implemented on a state-level that collects data about dispensed prescription drugs within the state. Health authorities, such as research organizations, government regulatory and law enforcement agencies, and medical clinicians and pharmacists maintain and access systems that store PMP data to track controlled substance prescriptions within the respective state. PMP data can provide timely information about prescribing and patient behaviors that may contribute to an epidemic and facilitate a targeted response.

Generally, medical practices that handle controlled substances submit controlled substance activity (e.g., prescriptions, dispensing, and the like) as a report to a state's PMP database to comply with government regulations. Because PMPs are implemented at state-level, reporting requirements may differ from state to state, such as in the types of data to be reported, the frequency at which PMP-related data must be reported, whether individual patient data should be reported, whether aggregate data can be reported, and so on. Heterogeneous data reporting requirements adds great complexity to identifying relevant data and ensuring that the data is in a format able to be processed by relevant systems. Further complicating the process is that typically, PMP-related data is manually recorded in a medical practice's physical logbooks and practice management systems.

### SUMMARY

Embodiments presented herein disclose a system and techniques for tracking and prescription drug monitoring program (PMP) data.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 illustrates a simplified conceptual diagram of a computing environment in which PMP-related data is identified and tracked within a digital controlled substance logbook platform of a medical practice;

FIG. 2 is a simplified block diagram of an example embodiment of the client device of FIG. 1;

FIG. 3 is a simplified block diagram of an example embodiment of the digital logbook platform server of FIG. 1;

FIG. 4 is a simplified flow diagram of a method for configuring a digital logbook platform application to identify and track PMP-related data within a digital logbook;

FIGS. 5A and 5B are example user interface views for configuring a digital logbook application of a client device to identify PMP-related data within a digital logbook;

FIG. 6 is a simplified flow diagram of a method for identifying and tracking PMP-related data within a digital logbook;

FIGS. 7A and 7B are example user interface views provided on a client device for identifying and tracking PMP-related data within a controlled log record of a digital logbook;

FIG. 8 is a simplified flow diagram of a method for generating a PMP data report for submission to a state PMP system; and

FIG. 9 is an example user interface view of a PMP report interface within the client device.

### DETAILED DESCRIPTION

Embodiments presented herein disclose systems and techniques for tracking, managing, and generating reports for “prescription monitoring program” (PMP) (also referred to as “prescription drug monitoring program” (PDMP)) data within a digital controlled substance logbook platform. More particularly, embodiments provide a digital controlled substance logbook platform that tracks any relevant or user-defined PMP data over the course of operation of the medical practice in managing controlled substances (e.g., inventoried, dispensed, prescribed, etc.).

The digital logbook provided by the platform enables a medical practice, e.g., via a client device accessing the platform through a graphical user interface, to enter PMP-relevant data, such as standardized usage data, over the day-to-day operation of the medical practice, e.g., while treating patients. The logbook platform may also obtain, aggregate, and standardize PMP-relevant data obtained from the medical practice's practice inventory management system (PIMS) to more efficiently validate data sets between the platform controlled substance usage logbook and the PIMS data. Advantageously, the platform serves as a verifiable source of truth for controlled substance management activity and thus ensures a complete and accurate set of data for complying with state PMP reporting requirements.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

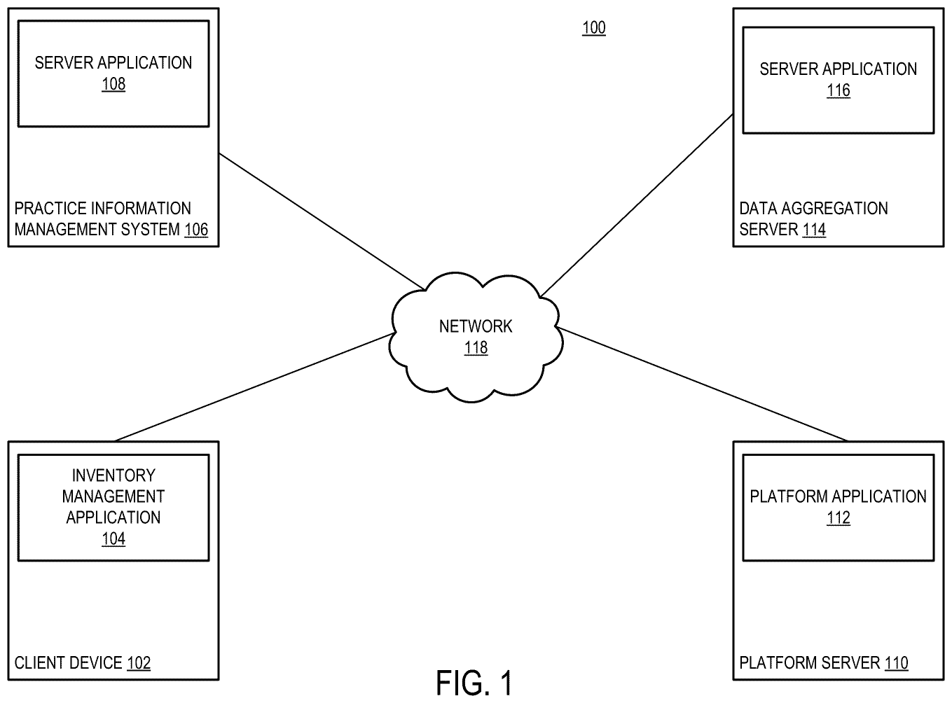

Referring now to FIG. 1, a computing environment 100 in which PMP-related data is tracked within a controlled substance log platform is now shown, according to an embodiment. As shown, the computing environment 100 includes a client device 102, a practice information management system (PIMS) 106, a platform server 110, and a data aggregation server 114, each interconnected via a network 118 (e.g., the Internet, a local area network, private network, etc.).

The client device 102 may be embodied as a physical computing device (e.g., a desktop computer, laptop computer, mobile device such as a mobile tablet or a smartphone, etc.) or a virtual computing instance executing in a cloud network. A user of the client device 102 may be an individual employed at a medical practice, such as a veterinary practice (e.g., a veterinary clinic, hospital, office with a pharmacy, etc.) that administers treatment to an animal patient, such as dispensing and administering medication to the patient, including controlled substances. As used herein, a controlled substance pertains to a drug or a chemical that is subject to governmental regulations with regard to manufacture, possession, and use.

Illustratively, the client device 102 includes an inventory management application 104. The inventory management application 104 provides a graphical user interface for drug inventory management enabling the user to record controlled substance usage over the course of the day. The interface may be embodied as a “digital logbook” that maintains records of usage and controlled substance inventory of the practice. For example, the user may, via the inventory management application 104, record entries of controlled substance usage into the digital log book provided by the interface. The interface may allow the user to enter, for example, patient information (e.g., name, chart ID, owner name, treatment), drug name, quantity, physician information, approval information, and so on. Further, the inventory management application 104 is integrated with one or more PIMS, such as PIMS 106.

The PIMS 106 may be a physical computing system (e.g., a desktop computer, workstation computer, laptop computer, etc.) or a virtual computing instance executing in the cloud. The PIMS 106 can also run on the premises of the veterinary practice or a remote location. As shown, the PIMS 106 further includes a server application 108, providing known functionalities of a PIMS, including managing patient data (including maintaining records of controlled substance usage per patient), invoicing, inpatient care workflows, reporting workflows, and so on.

In an embodiment, the inventory management application 104 is incorporated into an inventory management platform that includes one or more of the platform server 110 and the data aggregation server 114. In some embodiments, the data aggregation server 114 is a third-party server integrated with one or more components of the platform that retrieves, via the server application 116, PIMS data from the server application 108 and standardizes the data into a format that the inventory management application 104 can more easily interpret and further process. The server application 116 may integrate with the server application 108 of the PIMS 106 (e.g., via a module corresponding to the server application 116 incorporated into the server application 108). Further, the server application 116 may provide an application programming interface (API) to the platform application 112, allowing the platform application 112 to communicate and access functionalities provided by the data aggregation server 114. In other embodiments, one or a combination of the functions performed by the inventory management application 104, platform application 112, and server application 116 can be performed by a single application.

In an embodiment, the platform server 110 stores and maintains standardized PIMS data for later processing by the inventory management application 104. To do so, the platform server 110 includes a platform application 112 that performs such functions. The platform application 112 may provide an API to the inventory management application 104 to enable communication (e.g., transmission of PIMS data, local controlled usage log data, etc.) between the applications 104 and 112. In other embodiments, the platform application 112 also maintains and stores usage data provided by multiple client devices 102 each executing an instance of the inventory management application 104 (e.g., in the event that a client device 102 is a mobile device such as a tablet, and the inventory management application 104 is operated by multiple users. The platform application 112 may use APIs provided by the server application 116 to communicate with the PIMS 106.

As stated and as further described herein, controlled substance usage data may be recorded and maintained by the various components of the computing environment 100, and such data may be collected or tracked for subsequent reporting to a state PMP database. For example, the inventory management application 104 may collect data from transactional records that can include a date and time of the transaction, a name of the drug, the transaction activity, quantity of the drug involved in the transaction, patient or client information, prescribing doctor, pharmacist information, medical practice name and address, and the like. Similarly, the platform application 112 may collect data relating to reportable events for a state PMP database identifiable information such as drug name, date of usage, client, or patient chart number, and other typically recorded data. Once collected, the platform application 112 (e.g., through the inventory management application 104) may identify relevant data for submission to a state PMP data system based on requirements by the system and generate a compliant, structured, and compatible PMP report for submission (e.g., to a website associated with the PMP).

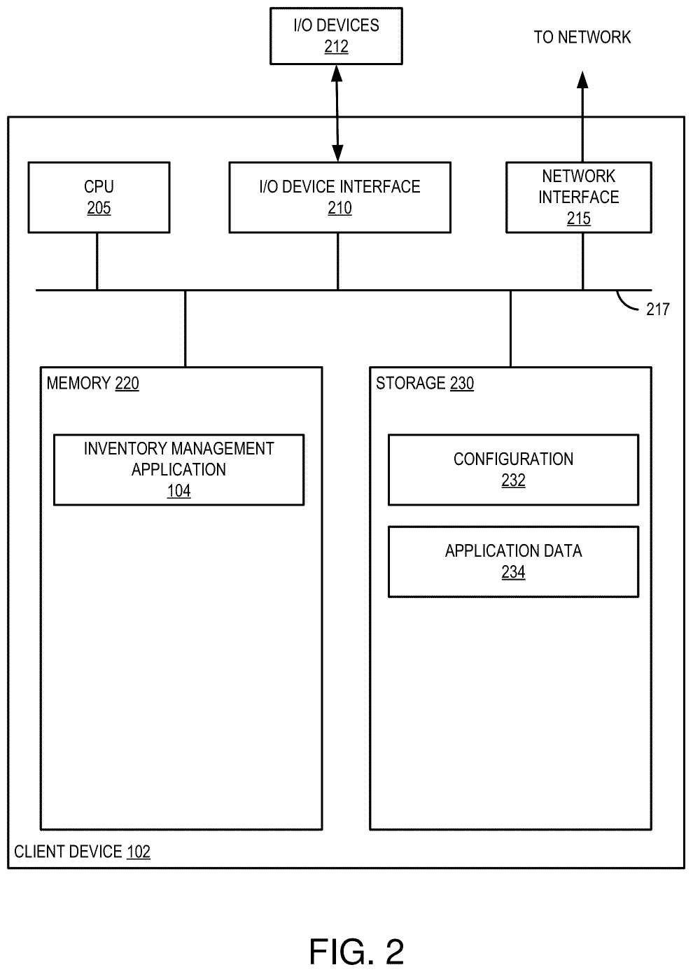

FIG. 2 further illustrates the client device 102. As shown, client device 102 includes, without limitation, a central processing unit (CPU) 205, an input/output (I/O) device interface 210, one or more I/O devices 212, a network interface 215, a memory 220, and a storage 230, each interconnected via a hardware bus 217. Of course, the actual client device 102 will include a variety of additional hardware components not shown. Additionally, in some embodiments, one or more of the illustrative components may be incorporated in, or otherwise form a portion of, another component.

The CPU 205 retrieves and executes programming instructions stored in the memory 220 (e.g., of the inventory management application 104). The CPU 205 may be embodied as one or more processors, each processor being a type capable of performing the functions described herein. For example, the CPU 205 may be embodied as a single or multi-core processor(s), a microcontroller, or other processor or processing/controlling circuit. In some embodiments, the CPU 205 may be embodied as, include, or be coupled to a field programmable gate array (FPGA), an application-specific integrated circuit (ASIC), reconfigurable hardware or hardware circuitry, or other specialized hardware to facilitate performance of the functions described herein. The hardware bus 217 is used to transmit instructions and data between the CPU 205, storage 230, network interface 215, and the memory 220. CPU 205 is included to be representative of a single CPU, multiple CPUs, a single CPU having multiple processing cores, and the like. The memory 220 may be embodied as any type of volatile (e.g., dynamic random access memory, etc.) or non-volatile memory (e.g., byte addressable memory) or data storage capable of performing the functions described herein. Volatile memory may be a storage medium that requires power to maintain the state of data stored by the medium. Non-limiting examples of volatile memory may include various types of random access memory (RAM), such as DRAM or static random access memory (SRAM). One particular type of DRAM that may be used in a memory module is synchronous dynamic random access memory (SDRAM). In particular embodiments, DRAM of a memory component may comply with a standard promulgated by JEDEC, such as JESD79F for DDR SDRAM, JESD79-2F for DDR2 SDRAM, JESD79-3F for DDR3 SDRAM, JESD79-4A for DDR4 SDRAM, JESD209 for Low Power DDR (LPDDR), JESD209-2 for LPDDR2, JESD209-3 for LPDDR3, and JESD209-4 for LPDDR4. Such standards (and similar standards) may be referred to as DDR-based standards and communication interfaces of the storage devices that implement such standards may be referred to as DDR-based interfaces.

The network interface 215 may be embodied as any hardware, software, or circuitry (e.g., a network interface card) used to connect the client device 102 over the network 118 and providing the network communication component functions described above. For example, the network interface 215 may be embodied as any communication circuit, device, or collection thereof, capable of enabling communications over the network 118 between the client device 102 and other devices (e.g., the PIMS 106, platform server 110, data aggregation server 114, etc.). The network interface 215 may be configured to use any one or more communication technology (e.g., wired, wireless, and/or cellular communications) and associated protocols (e.g., Ethernet, Bluetooth®, Wi-Fi®, WiMAX, 5G-based protocols, etc.) to effect such communication. For example, to do so, the network interface 215 may include a network interface controller (NIC, not shown), embodied as one or more add-in-boards, daughtercards, controller chips, chipsets, or other devices that may be used by the computing device 102 for network communications with remote devices. For example, the NIC may be embodied as an expansion card coupled to the I/O device interface 210 over an expansion bus such as PCI Express.

The I/O device interface 210 allows the I/O devices 212 to communicate with hardware and software components of the client device 102. For example, the I/O device interface 210 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O device interface 210 may form a portion of a system-on-a-chip (SoC) and be incorporated, along with one or more of the CPU 205, the memory 220, and other components of the client device 102.

The I/O devices 212 may be embodied as any type of I/O device connected with or provided as a component to the client device 102. I/O devices such as keyboards, mice, and printers may be included as I/O devices 212 (e.g., to enter controlled substance usage information once administered to a patient). Illustratively, the memory 220 includes the inventory management application 104.

The storage 230 may be embodied as any type of devices configured for short-term or long-term storage of data such as, for example, memory devices and circuits, memory cards, hard disk drives (HDDs), solid-state drives (SSDs), or other data storage devices. The storage 230 may include a system partition that stores data and firmware code for the storage 230. The storage 230 may also include an operating system partition that stores data files and executables for an operating system.

As shown, the storage 230 includes a configuration 232 and application data 234. The configuration 232 may be embodied as any data indicative of user-definable parameters and state-by-state requirements for managing the PMP-data collection process. Generally, the configuration 232 defines relevant fields for collection from transaction records generated by the inventory management application 104 and/or the platform application 112. The defined relevant fields may correspond to those required by a given state as well as optional user-defined fields. The application data 234 may be embodied as any type of data generated or maintained by the inventory management application 104, such as controlled substance usage data.

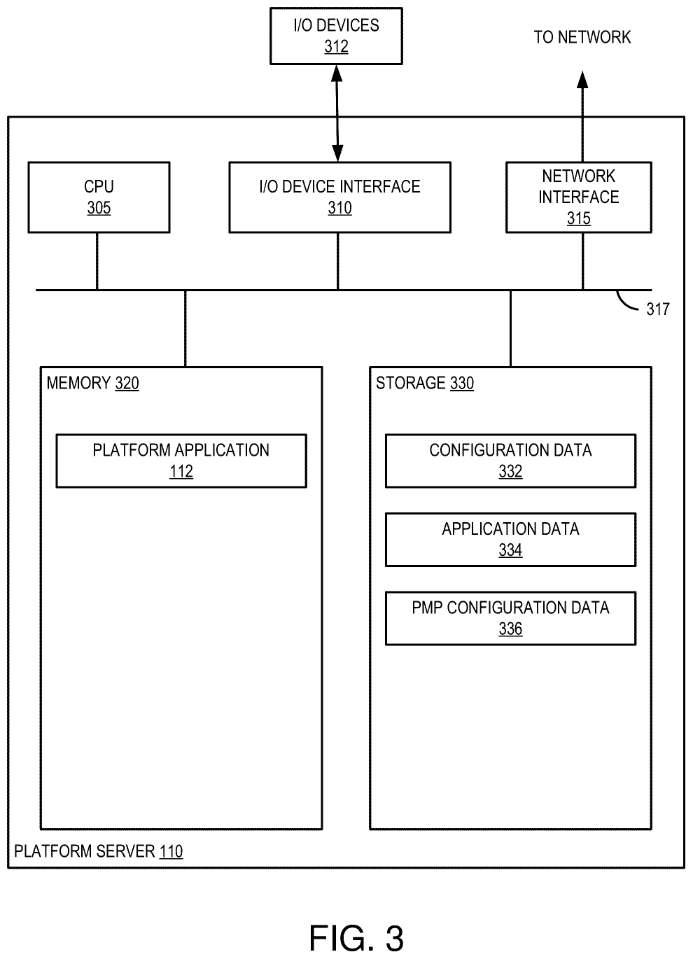

FIG. 3 further illustrates the platform server 110. As shown, the platform server 110 includes, without limitation, a CPU 305, an I/O device interface 310, one or more I/O devices 312, and a network interface 315, memory 320, and storage 330, each interconnected via a hardware bus 317. Of course, the actual platform server 110 will include a variety of additional hardware components not shown. Additionally, in some embodiments, one or more of the illustrative components may be incorporated in, or otherwise form a portion of, another component. The CPU 305, I/O device interface 310, I/O devices 312, network interface 315, hardware bus 317, memory 320, and storage 330 are similar to the respective CPU 205, I/O device interface 210, I/O devices 212, network interface 215, hardware bus 217, memory 220, and storage 230 of the client device 102. Illustratively, the memory 320 includes the platform application 112. The storage 330 further includes configuration data 332, which may be embodied as any data indicative of user-definable parameters and rules for managing controlled substance usage data collection and tracking. The storage 330 also includes application data 334, which may be embodied as any type of data relating to standardized controlled substance usage data retrieved from the data aggregation server 114. In other embodiments, the application data 334 may include controlled substance usage logs sent by the inventory management application 104. The storage 330 also includes PMP configuration data 336, which corresponds to a global configuration file defining fields to capture and a structure for which to format the fields for a PMP report submission, e.g., for acceptance by a state PMP system.

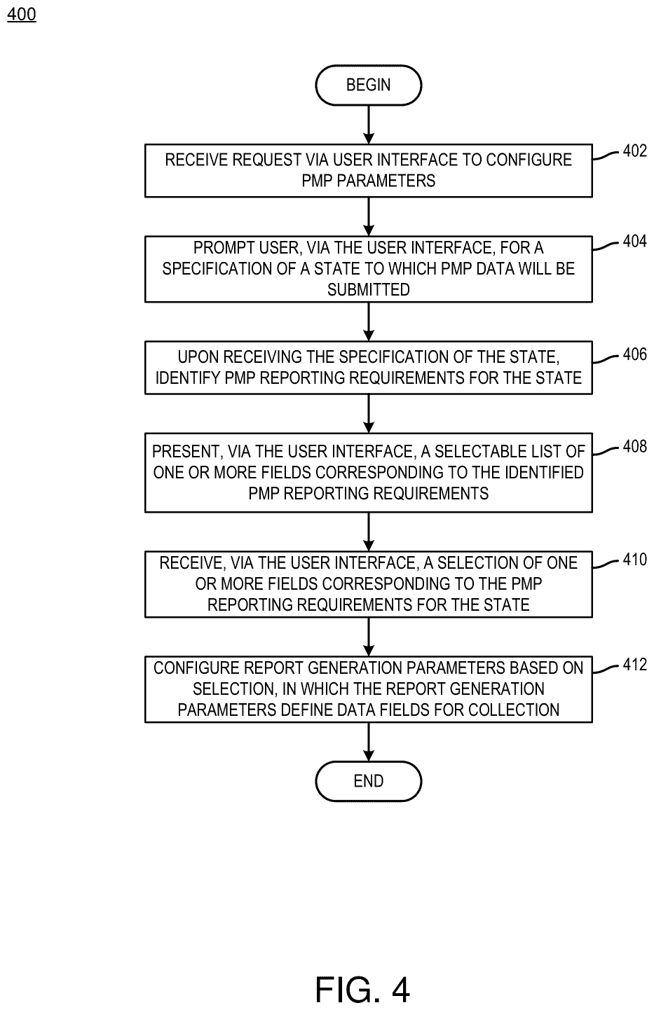

Referring now to FIG. 4, the inventory management application 104, in operation, may perform a method 400 for configuring controlled substance usage collection to identify and track PMP-related data. Method 400 relates to an initial setup for configuring the inventory management application 104 to identify, collect, and track PMP data. In an embodiment, the inventory management application 104 may use a configuration based on an ASAP (American Society for Automation in Pharmacy) file format required by a state. Generally, ASAP standards and format are used by states that have a prescription monitoring program. For example, ASAP version 4.1 defines all required and optional fields in generation and submission. The ASAP version number contains all fields that are available, and each state may determine which version number it will currently accept. Each state also determines which fields (e.g., a subset of fields from the ASAP version number) that are actually required. For example, the state of California may require a few or more fields than the currently accepted ASAP version number.

The method 400 begins in block 402, in which the inventory management application 104, via a user interface, receives a request to configure PMP fields for collection. In block 404, the inventory management application 104 prompts the user via the interface for a specification of a state to which PMP data will be submitted. In an embodiment, the inventory management application 104 may also prompt the user for a specification of an ASAP version number for submission to the state.

Upon receiving the specification of the state, in block 406, the inventory management application 104 identifies PMP reporting requirements for the state. For example, the inventory management application 104 may prompt the user for a specification of fields corresponding to reporting requirements. In some embodiments, the inventory management application 104 may connect to a state's PMP system (e.g., via a website or an API provided by the state's PMP system) to obtain reporting requirements and parse the requirements (e.g., via a natural language processing algorithm, API, etc.) to identify the fields. Further, the inventory management application 104 may store previously identified PMP reporting requirements and respective fields locally or obtain the requirements from the platform application. In block 408, the inventory management application presents, via the user interface, a selectable list of one or more fields corresponding to the identified PMP reporting requirements. The list of fields can include a pharmacy identifier, DEA identifier, quantity information, drug name information, date and time information, and the like. The user interface may instruct the user to select one or more of the fields on the list for collection. In an embodiment, the inventory management application 104 prompts the user to select fields based on the ASAP version previously specified. For example, the inventory management application 104 may filter out fields that are inapplicable to the specified ASAP version and present the remaining fields for selection by the user via the graphical user interface. In block 408, the inventory management application 104 receives the selection of one or more of the fields.

In block 410, the inventory management application 104 configures report generation parameters based on the selection. The inventory management application 104 may automatically generate header and trailer sections for the ASAP file containing all necessary PMP data for reporting to the selected state. Further, the inventory management application 104 may populate static fields typically included in a PMP report (e.g., hospital name, address, etc.) into the configured report generation parameters, such that this information is generated in each resulting PMP report. The medical practice user may refer to a PDF file (e.g., linked by the inventory management application 104) while also configuring desired data fields for collection.

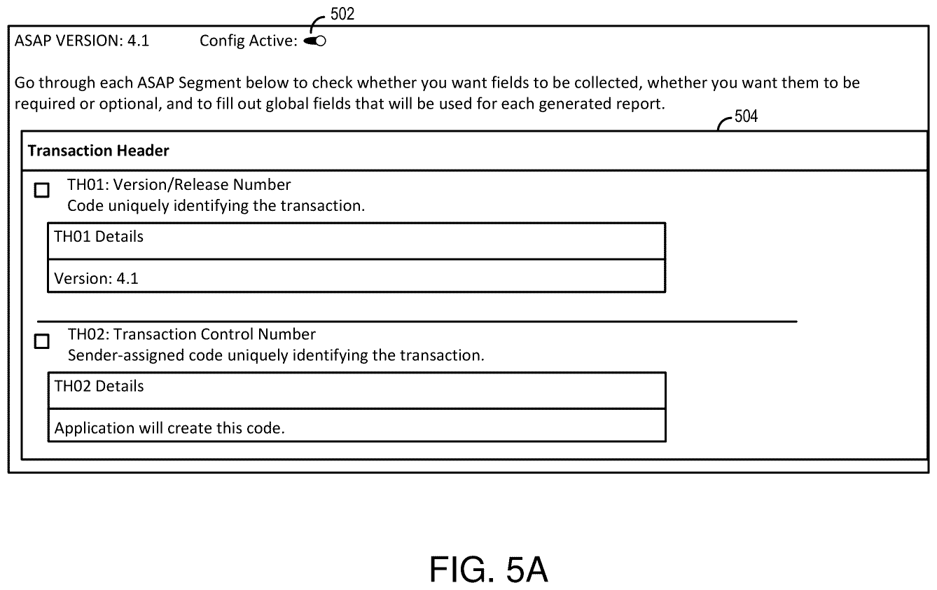

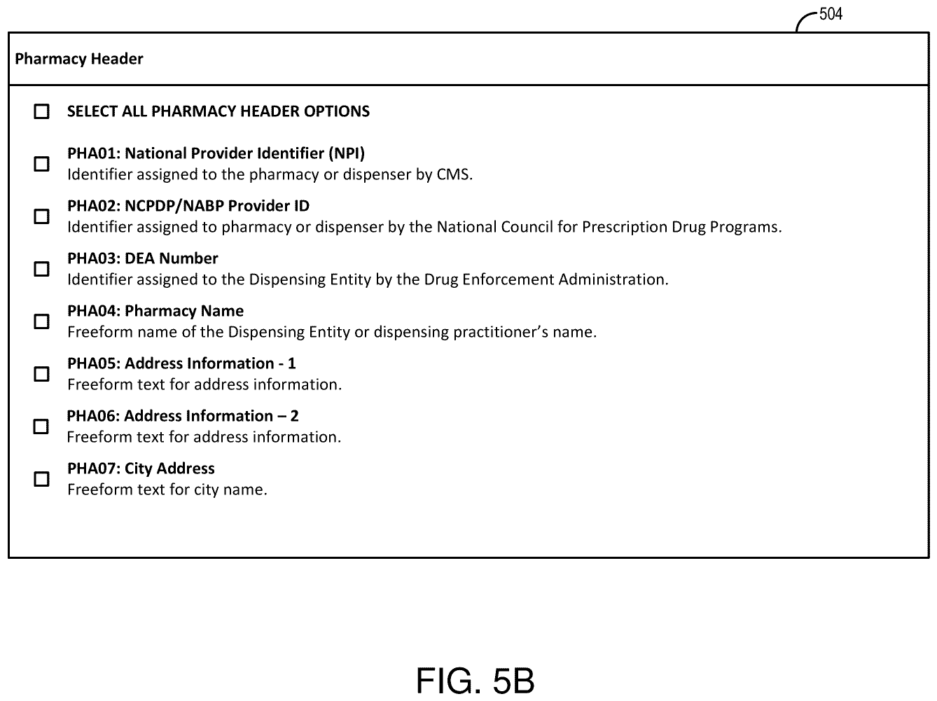

FIGS. 5A and 5B illustrate example graphical user interface views of the inventory management application 104 in which the ASAP file can be configured. As shown in FIG. 5A, the user interface enables the user to configure fields to be collected for ASAP version 4.1. The user may toggle whether the inventory management application 104 collects data for the ASAP version 4.1 using a toggle widget 502. Through the user interface, the user may scroll through segments 504 (FIG. 5A displaying a segment 504 for the transaction header and FIG. 5B displaying a segment 504 for the pharmacy header) and select, e.g., via checkboxes, which fields under the relevant segments to include. In an embodiment, the user interface may provide a brief description associated with each field. For example, in FIG. 5A, under the field TH01 for version and release number, the user interface provides associated information that the field relates to a code uniquely identifying an underlying transaction. Under the field TH02 for transaction control number, the user interface provides associated information that the field relates to a sender-assigned code uniquely identifying the underlying transaction. The user interface, as shown in FIG. 5A, also provides further details relating to each field relevant to how the field will be collected and provided on a PMP report. For example, for TH01, a details text box indicates that the version release number for the transaction will be collected as ASAP version 4.1, and for TH02, a details text box indicates that the control number provided will be generated and stored by the inventory management application 104.

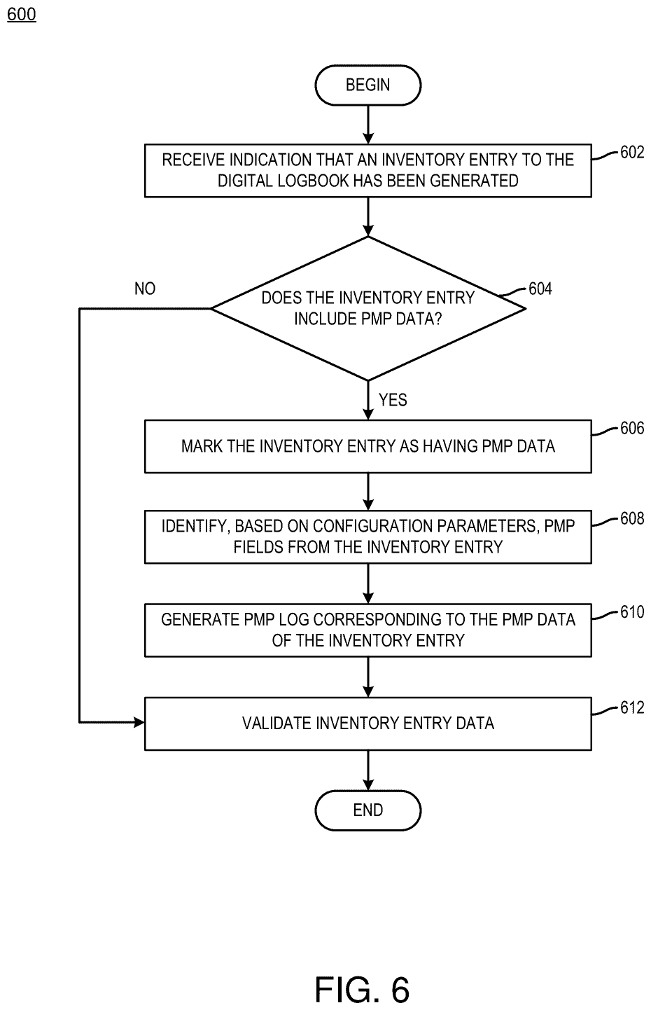

Once configured for data collection, the inventory management application 104 may mark records as including PMP data when creating controlled drug log information therein, as well as collect additional information as specified by the medical practice user. Referring now to FIG. 6, a method 600 for identifying PMP-related data within a digital logbook is shown. In block 602, the inventory management application 104 receives an indication that an entry of data into the digital logbook has been generated (e.g., a pharmacist for the medical practice records the dispensing of a controlled substance to a patient).

In block 604, the inventory management application 104 determines whether any of the fields correspond to PMP-related data fields based on the previously generated configuration of method 400. More particularly, the inventory management application 104 evaluates the entry to determine whether the entry includes fields (previously specified by the user for collection) to be included with a PMP data report. If not, then the method 600 ends. However, if so, then in block 606, the inventory management application 104 marks the inventory entry as including PMP data. For example, the inventory management application 104 may append data to the entry indicating that the entry includes PMP data. In some embodiments, each entry may include a binary field that can be toggled to indicate that the inventory entry includes PMP data.

In block 608, the inventory management application 104 identifies, based on the previously generated configuration parameters, PMP fields from the inventory entry. In block 610, the inventory management application 104 generates a PMP log that incorporates the identified fields corresponding to the PMP data. The medical practice may collect such information via the digital logbook and during reconciliation (e.g., while the medical practice is still able to edit the controlled log after creation). In block 612, the inventory management application 104 validates the controlled log entry data to ensure any currently required information is entered.

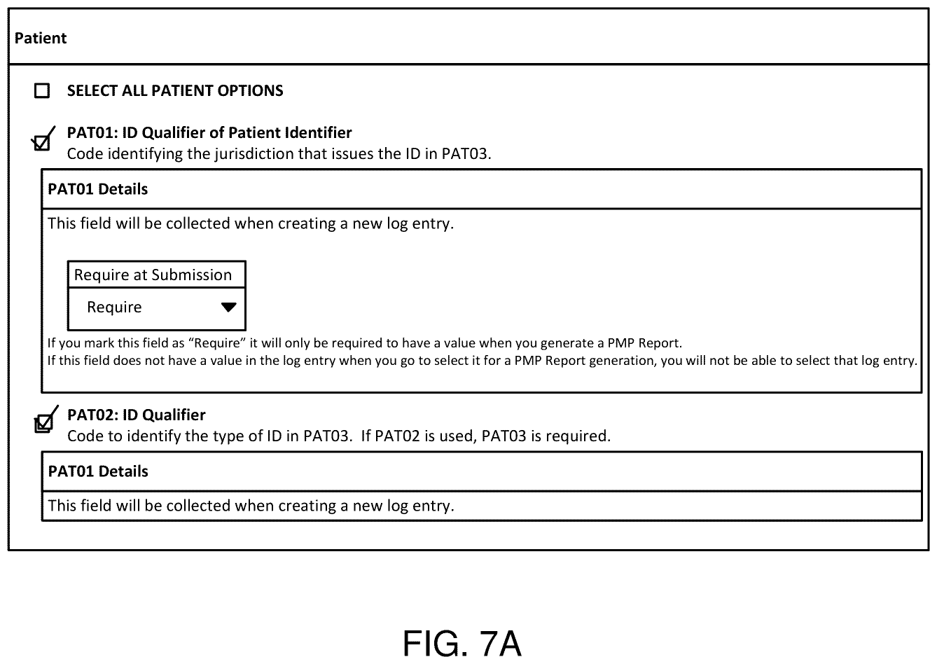

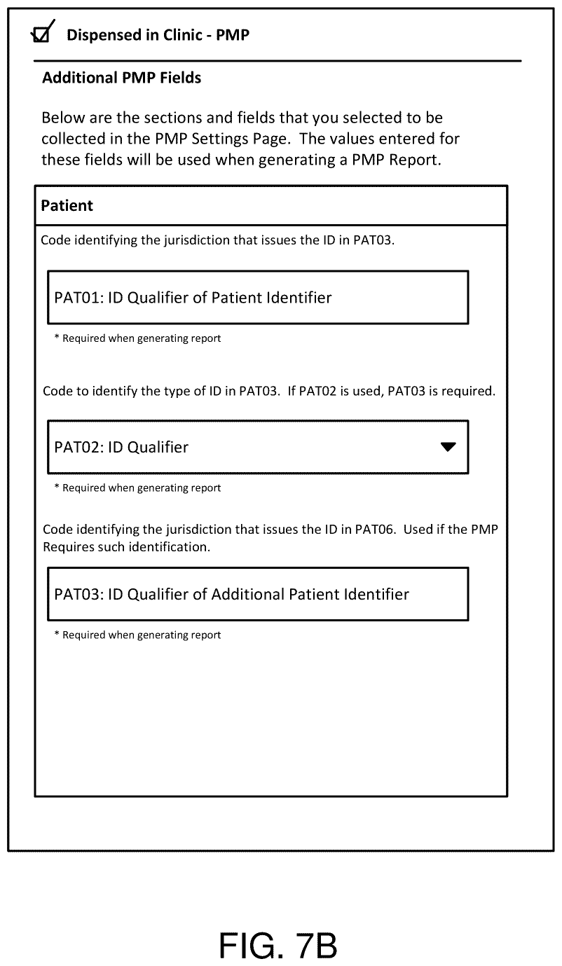

FIGS. 7A and 7B illustrate example graphical user interface views of identifying PMP-related data in an inventory log entry. More particularly, inventory management application 104 may incorporate such views in the inventory entry creation process (e.g., for a controlled substance). Advantageously, integrating these views for identifying PMP within an inventory account for the overlap of controlled substance recordation requirements and PMP-reporting requirements to create a more streamlined and efficient inventory entry process. The user (e.g., a veterinary practice personnel) may indicate, during entry of the controlled substance, which fields to collect and incorporate in a PMP log separate from the digital logbook and which fields to filter out during creation. For example, FIG. 7A illustrates a user interface view in which the user may indicate, using checkboxes, which patient-related fields to include in a PMP log during creation of an inventory entry. The user interface view may also include information and additional parameters for each field. For example, under the field for PAT01 for an ID qualifier of a patient identifier, a dropdown box is included to allow the user to indicate whether to require the PAT01 to have a value when a PMP report is generated. The user interface view of FIG. 7B presents, to the user, additional ields previously indicated by the user to include in a PMP report to provide for case of use and understanding for the user.

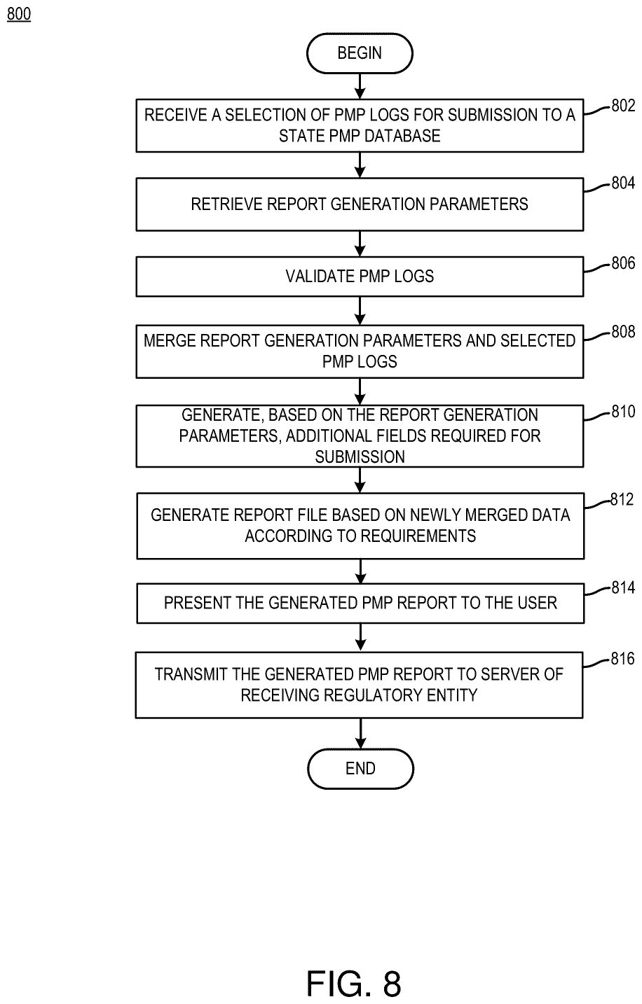

The medical practice, at required or desired intervals, may generate, from the collected PMP data, a PMP report of records for submission to a state PMP database. Referring now to FIG. 8, a method 800 for generating a PMP data report from collected is shown. In block 802, the inventory management application 104 receives a selection of PMP logs for submission to a database, e.g., following a request via the user interface to generate a PMP report. In block 804, the inventory management application 104 retrieves, from the digital logbook, data that was previously marked for PMP submission. In some embodiments, the inventory management application 104 may store the PMP-related data in a separate data store for more efficient retrieval of the data, compared to other prior approaches of iterating through PIMS data to identify PMP-relevant data.

In block 806, the inventory management application 104 validates the PMP logs to ensure completeness and accuracy of the information entered therein. In an embodiment, the inventory management application 104 may identify errors or inaccuracies and reconcile the data based on data from different sources (e.g., third-party sources, the PIMS 106, etc.). Further in an embodiment, the inventory management application 104 may prompt the user to include any missing data (e.g., data that was not included at the time of the creation of a given PMP log, such as days supply information) or confirm the accuracy of the data. In block 808, the inventory management application 104 merges the report generation parameters with the selected PMP logs to create an intermediate report file. For example, the report generation parameters may include static data that is typically provided in a PMP report, such as hospital information (name, address, identifier). The intermediate report file incorporates such static data as well as the PMP log data. Once generated, the inventory management application 104 may further evaluate the intermediate report to determine whether any additional fields are required for submission.

In block 810, the inventory management application 104 populates the additional fields into the intermediate report and respective data (and/or prompts the user for any missing data for those additional fields). In block 812, the inventory management application 104 finalizes the PMP report according to state requirements and the PMP configuration parameters. In an embodiment, once generated, the inventory management application 104 may unmarked the previously marked data (such that duplicate data is not entered in subsequent reports). In block 814, the inventory management application 104 presents the generated PMP report to the user, e.g., in a file format required by the underlying state (e.g., in PDF format). The user may thereafter submit the generated report to the receiving regulatory entity system, such as a state PMP database system. In other embodiments, for example, in block 816, the inventory management application 104 may directly submit the generated report to the receiving regulatory entity system.

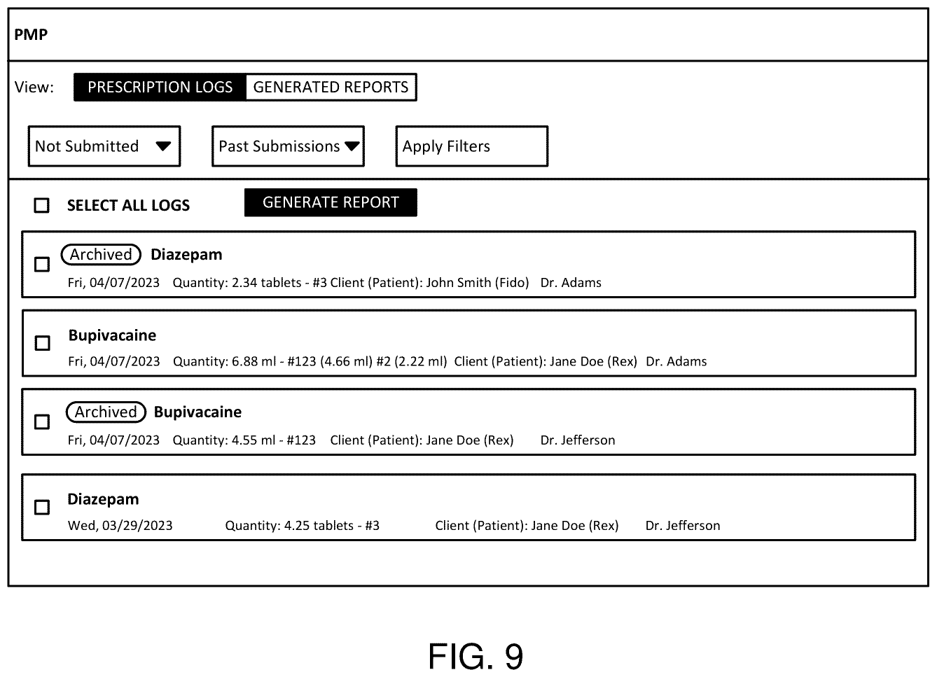

FIG. 9 provides a user interface view of a PMP report generation in which a user may generate a PMP report compiling collected data that is required for submission to a receiving regulatory entity system, such as a state PMP database system. The inventory management application 104 retrieves all records for a given period for entering into a report. In an embodiment, the inventory management application 104 may automatically determine whether records have already been reported to the state (e.g., using an indicator to associate with a record, in which the indicator signifies that data in the record has been submitted to a state regulatory entity).

The graphical user interface of the inventory management application 104 guides a user through the process of selecting relevant subsets of PMP fields, given the complexities involved in different states requiring different subsets of PMP fields (in which some states have mandatory fields that may be discretionary fields in other states). As shown, the user interface allows the user to alternate views between logged prescription data and previously generated reports (here, logged transactions for controlled substances are currently shown in the view). The inventory management application 104 may generate PMP entries from select transactions. More particularly, each PMP entry is from a digital logbook entry (e.g., of a controlled substance), but not every digital logbook entry might correspond to a PMP submission. The user interface shown in FIG. 9 lists log entries for transactions of controlled substances, such as for diazepam and bupivacaine, occurring at different dates, for different substance quantities, clients, patients, and medical professionals at a veterinary practice. Once the user clicks the Generate Report button (of FIG. 9) on the user interface, the inventory management application 104 may validate each PMP entry. For instance, to do so, the inventory management application 104 may ensure that the log entry is finalized and filled properly (e.g., no required fields are missing, no inconsistencies are present in quantities relative to a drug container, etc.). Once generated, the resulting report will be in a format (e.g., an ASAP formatted file) that is acceptable by a regulatory entity system.

While the foregoing is directed to embodiments of the present disclosure, other and further embodiments of the disclosure may be devised without departing from the basic scope thereof, and the scope thereof may be determined by the example claims that follow.

### EXAMPLES

Illustrative examples of the technologies disclosed herein are provided below. An embodiment of the technologies may include any one or more, and any combination of, the examples described below.

Example 1 includes a computer-implemented method for managing prescription drug monitoring program (PMP) data, the method comprising receiving, by a client device associated with a platform application, a request to generate a report of PMP data maintained by a medical practice, the report to be submitted to a regulatory entity system of a given state; identifying, by the client device, one or more data requirements for the report associated with the state; obtaining, by the client device and from a digital controlled substance logbook maintained by the platform application, one or more records including data that complies with the one or more data requirements; extracting, by the client device, the data that complies with the one or more data requirements from the records; and generating, by the client device, the PMP report from the extracted data.

Example 2 includes the subject matter of Example 1, and further including storing the one or more records including the data that complies with the one or more data requirements in a separate log file.

Example 3 includes the subject matter of any of Examples 1 and 2, and further including transmitting the PMP report to the regulatory entity system.

Example 4 includes the subject matter of any of Examples 1-3, and wherein the medical practice is a veterinary practice.

Example 5 includes the subject matter of any of Examples 1-4, and wherein the one or more data requirements is obtained from the regulatory entity system.

Example 6 includes the subject matter of any of Examples 1-5, and further including entering a transactional record into the digital controlled substance logbook; and upon determining that the transactional record includes data required to be submitted as part of the PMP report, associating the transactional record with an indicator for submission to the PMP report.

Example 7 includes the subject matter of any of Examples 1-6, and further including validating the extracted data that complies with the one or more data requirements.

Example 8 includes a system comprising one or more processors; and a memory storing a plurality of instructions, which, when executed on the one or more processors, causes the system to receive a request to generate a report of prescription drug monitoring program (PMP) data maintained by a medical practice, the report to be submitted to a regulatory entity system of a given state, identify one or more data requirements for the report associated with the state, obtain, from a digital controlled substance logbook, one or more records including data that complies with the one or more data requirements, extract the data that complies with the one or more data requirements from the records, and generate the PMP report from the extracted data.

Example 9 includes the subject matter of Example 8, and wherein the plurality of instructions further causes the system to store the one or more records including the data that complies with the one or more data requirements in a separate log file.

Example 10 includes the subject matter of any of Examples 8 and 9, and wherein the plurality of instructions further causes the system to transmit the PMP report to the regulatory entity system.

Example 11 includes the subject matter of any of Examples 8-10, and wherein the medical practice is a veterinary practice.

Example 12 includes the subject matter of any of Examples 8-11, and wherein the one or more data requirements is obtained from the regulatory entity system.

Example 13 includes the subject matter of any of Examples 8-12, and wherein the plurality of instructions further causes the system to enter a transactional record into the digital controlled substance logbook; and upon a determination that the transactional record includes data required to be submitted as part of the PMP report, associate the transactional record with an indicator for submission to the PMP report.

Example 14 includes the subject matter of any of Examples 8-13, and wherein the plurality of instructions further causes the system to validate the extracted data that complies with the one or more data requirements.

Example 15 includes a machine-readable storage medium storing a plurality of instructions, which, when executed on one or more processors, causes a system to receive a request to generate a report of prescription drug monitoring program (PMP) data maintained by a medical practice, the report to be submitted to a regulatory entity system of a given state; identify one or more data requirements for the report associated with the state; obtain, from a digital controlled substance logbook, one or more records including data that complies with the one or more data requirements; extract the data that complies with the one or more data requirements from the records; and generate the PMP report from the extracted data.

Example 16 includes the subject matter of Example 15, and wherein the plurality of instructions further causes the system to store the one or more records including the data that complies with the one or more data requirements in a separate log file.

Example 17 includes the subject matter of any of Examples 15 and 16, and wherein the plurality of instructions further causes the system to transmit the PMP report to the regulatory entity system.

Example 18 includes the subject matter of any of Examples 15-17, and wherein the one or more data requirements is obtained from the regulatory entity system.

Example 19 includes the subject matter of any of Examples 15-18, and wherein the plurality of instructions further causes the system to enter a transactional record into the digital controlled substance logbook; and upon a determination that the transactional record includes data required to be submitted as part of the PMP report, associate the transactional record with an indicator for submission to the PMP report.

Example 20 includes the subject matter of any of Examples 15-19, and wherein the plurality of instructions further causes the system to validate the extracted data that complies with the one or more data requirements.

We claim:

1. A computer-implemented method for managing prescription drug monitoring program (PMP) data, the method comprising:
receiving, by a client device associated with a platform application, a request to generate a report of PMP data maintained by a medical practice, the report to be submitted to a regulatory entity system of a given state;
identifying, by the client device, one or more data requirements for the report associated with the state;
obtaining, by the client device and from a digital controlled substance logbook maintained by the platform application, one or more records including data that complies with the one or more data requirements;
extracting, by the client device, the data that complies with the one or more data requirements from the records; and
generating, by the client device, the PMP report from the extracted data.

2. The computer-implemented method of claim 1, further comprising:
storing the one or more records including the data that complies with the one or more data requirements in a separate log file.

3. The computer-implemented method of claim 1, further comprising:
transmitting the PMP report to the regulatory entity system.

4. The computer-implemented method of claim 1, wherein the medical practice is a veterinary practice.

5. The computer-implemented method of claim 1, wherein the one or more data requirements is obtained from the regulatory entity system.

6. The computer-implemented method of claim 1, further comprising:
entering a transactional record into the digital controlled substance logbook; and
upon determining that the transactional record includes data required to be submitted as part of the PMP report, associating the transactional record with an indicator for submission to the PMP report.

7. The computer-implemented method of claim 1, further comprising:
validating the extracted data that complies with the one or more data requirements.

8. A system comprising:
one or more processors; and
a memory storing a plurality of instructions, which, when executed on the one or more processors, causes the system to:
receive a request to generate a report of prescription drug monitoring program (PMP) data maintained by a medical practice, the report to be submitted to a regulatory entity system of a given state,
identify one or more data requirements for the report associated with the state,
obtain, from a digital controlled substance logbook, one or more records including data that complies with the one or more data requirements,
extract the data that complies with the one or more data requirements from the records, and
generate the PMP report from the extracted data.

9. The system of claim 8, wherein the plurality of instructions further causes the system to:
store the one or more records including the data that complies with the one or more data requirements in a separate log file.

10. The system of claim 8, wherein the plurality of instructions further causes the system to:
transmit the PMP report to the regulatory entity system.

11. The system of claim 8, wherein the medical practice is a veterinary practice.

12. The system of claim 8, wherein the one or more data requirements is obtained from the regulatory entity system.

13. The system of claim 8, wherein the plurality of instructions further causes the system to:
enter a transactional record into the digital controlled substance logbook; and
upon a determination that the transactional record includes data required to be submitted as part of the PMP report, associate the transactional record with an indicator for submission to the PMP report.

14. The system of claim 8, wherein the plurality of instructions further causes the system to:
validate the extracted data that complies with the one or more data requirements.

15. A machine-readable storage medium storing a plurality of instructions, which, when executed on one or more processors, causes a system to:
receive a request to generate a report of prescription drug monitoring program (PMP) data maintained by a medical practice, the report to be submitted to a regulatory entity system of a given state;
identify one or more data requirements for the report associated with the state;
obtain, from a digital controlled substance logbook, one or more records including data that complies with the one or more data requirements;
extract the data that complies with the one or more data requirements from the records; and
generate the PMP report from the extracted data.

16. The system of claim 8, wherein the plurality of instructions further causes the system to:
store the one or more records including the data that complies with the one or more data requirements in a separate log file.

17. The system of claim 8, wherein the plurality of instructions further causes the system to:
transmit the PMP report to the regulatory entity system.

18. The system of claim 8, wherein the one or more data requirements is obtained from the regulatory entity system.

19. The system of claim 8, wherein the plurality of instructions further causes the system to:
enter a transactional record into the digital controlled substance logbook; and
upon a determination that the transactional record includes data required to be submitted as part of the PMP report, associate the transactional record with an indicator for submission to the PMP report.

20. The system of claim 8, wherein the plurality of instructions further causes the system to:
validate the extracted data that complies with the one or more data requirements.
